(19) United States
(12) Patent Application Publication
(10) Pub. No.: US 2022/0379077 A1
(43) Pub. Date: Dec. 1, 2022

(54) MEDICAL ELONGATED BODY

(71) Applicant: TERUMO KABUSHIKI KAISHA, Tokyo (JP)

(72) Inventors: Ryo OKAMURA, Fujinomiya (JP); Koki FUSE, Hadano (JP)

(73) Assignee: TERUMO KABUSHIKI KAISHA, Tokyo (JP)

(21) Appl. No.: 17/883,909

(22) Filed: Aug. 9, 2022

Related U.S. Application Data
(63) Continuation of application No. PCT/JP2021/009149, filed on Mar. 9, 2021.

(30) Foreign Application Priority Data
Mar. 27, 2020 (JP) 2020-058900

(51) Int. Cl. A61M 25/00; A61M 39/02

(52) U.S. Cl. CPC A61M 25/0045; A61M 39/0247; A61M 25/0097; A61M 2039/0291; A61M 2025/0059; A61M 2025/0046; A61M 2025/0057; A61M 2025/006; A61M 2025/0681

(57) ABSTRACT

A medical elongated body that is capable of improving convenience during use by a surgeon, and capable of preventing peeling, from a catheter main body, of a drug portion (a hydrophilic coating) containing metal ions and a drug when disposed on a wound site of a patient. An introducer sheath includes a tubular sheath configured to be percutaneously inserted into a blood vessel, and a hub connected to a proximal portion of the tubular sheath, in which the tubular sheath includes a first region and a second region located on a proximal side relative to the first region, the first region has a first hydrophilic coating having lubricity, the second region includes a groove portion extending from a distal side of the tubular sheath toward a proximal side of the tubular sheath, the groove portion having a second hydrophilic coating containing at least one of metal ions and a drug.

### CROSS-REFERENCES TO RELATED APPLICATIONS

This application is a continuation of International Application No. PCT/JP2021/009149 filed on Mar. 9, 2021, which claims priority to Japanese Patent Application No. 2020-058900 filed on Mar. 27, 2020, the entire content of both of which is incorporated herein by reference.

### TECHNOLOGICAL FIELD

The present disclosure generally relates to a medical elongated body.

### BACKGROUND DISCUSSION

In the related art, in a catheter procedure or the like, a medical elongated body is inserted into a living body lumen such as a blood vessel to examine and treat a lesion site. An example of the medical elongated body includes an introducer sheath that forms an access path connecting the outside of a living body and the living body lumen.

The introducer sheath includes a tubular sheath (a catheter main body) and a hub connected to the tubular sheath. In a procedure using the introducer sheath, a surgeon or the like percutaneously inserts the tubular sheath into the living body lumen (for example, the blood vessel) through a puncture site (puncture) formed in a limb or the like of a patient in a state in which a dilator is inserted into the tubular sheath. The surgeon or the like removes the dilator from the tubular sheath in a state in which a distal side of the tubular sheath is inserted into the blood vessel. After the dilator is removed from the tubular sheath, the surgeon or the like can introduce a guide wire, various catheter devices, or the like into the blood vessel through a lumen of the tubular sheath.

In recent years, as described in WO 2018/043427, a method has been disclosed in which hemostasis at a puncture site is promoted by disposing a hemostatic agent that enables wound healing on an outer surface of a tubular sheath, thereby shortening time required for the hemostasis at the puncture site of a patient and reducing a physical burden on the patient.

In the introducer sheath described in WO 2018/043427, the hemostatic agent is disposed on the outer surface of the tubular sheath. In the introducer sheath described in WO 2018/043427, the hemostatic agent is covered with a cover member or a strain relief in order to help prevent peeling of the hemostatic agent from the tubular sheath before the hemostatic agent is introduced to the puncture site.

As described above, in the introducer sheath described in WO 2018/043427, since the hemostatic agent disposed on the outer surface of the tubular sheath is covered with the strain relief or the cover member, the hemostatic agent can be prevented from coming into contact with fingers of a surgeon or the like or surrounding articles and getting lost due to scratching or the like. Therefore, after removing the cover member and exposing the hemostatic agent to the outer surface of the tubular sheath, the surgeon or the like needs to dispose the hemostatic agent on a wound site of the patient by operating the introducer sheath while being careful such that the hemostatic agent does not to come into contact with fingers or the surrounding articles.

### SUMMARY

A medical elongated body is disclosed that is capable of further improving convenience during use by a surgeon or the like, and capable of preventing peeling, from a catheter main body, of a drug portion (a hydrophilic coating) containing metal ions or a drug when the medical elongated body is disposed on a wound site of a patient.

The medical elongated body according to the disclosure includes a catheter main body configured to be percutaneously inserted into a living body lumen, and a hub connected to a proximal portion of the catheter main body, in which the catheter main body includes a first region and a second region located on a proximal side relative to the first region, the first region has a first hydrophilic coating having lubricity, the second region includes a groove portion extending from a distal side of the catheter main body toward a proximal side of the catheter main body, and the groove portion has a second hydrophilic coating containing at least one of metal ions and a drug.

In the medical elongated body described above, the second hydrophilic coating containing at least one of the metal ions and the drug is located in the groove portion formed in the second region of the catheter main body. When the catheter main body is inserted into a living body lumen (for example, a blood vessel), body fluid (for example, blood) enters the groove portion of the second region, and the second hydrophilic coating swells, and thus the medical elongated body can release the metal ions and the drug retained by the second hydrophilic coating to a living body tissue (for example, a subcutaneous tissue) and a blood vessel wall surrounding the catheter main body. The second hydrophilic coating is located in the groove portion of the second region. Therefore, when a surgeon or the like operates the medical elongated body, in the medical elongated body configured as described above, since fingers, surrounding articles, and the like do not come into contact with the second hydrophilic coating, the medical elongated body can prevent peeling, from the catheter main body, of the second hydrophilic coating that retains the metal ions and the drug. Accordingly, the medical elongated body can simplify an operation of disposing the second hydrophilic coating on a puncture site by the surgeon or the like, and thus the metal ions and the drug retained by the second hydrophilic coating can be easily provided on the puncture site of a patient. Since the second hydrophilic coating is located in the groove portion, the medical elongated body can prevent the catheter main body from slipping when the surgeon or the like grips the proximal side of the catheter main body with fingers or the like.

In addition, the medical elongated body can also prevent, when the surgeon or the like indwells the second region having the second hydrophilic coating at the puncture site, a movement of the catheter main body (sliding of the catheter main body due to the second hydrophilic coating) at an indwelling site. In the medical elongated body, flexibility of the catheter main body is improved by the groove portion provided in the second region of the catheter main body. Therefore, the medical elongated body can prevent a kink in the catheter main body while the catheter main body is indwelled in the blood vessel. In the medical elongated body, since the first region located on the distal side of the catheter main body relative to the second region has the first hydrophilic coating, the catheter main body can be smoothly inserted into the living body lumen, and the second hydrophilic coating located in the second region can be smoothly disposed in the puncture site.

In accordance with an aspect, an introducer sheath is disclosed comprising: a tubular sheath, the tubular sheath including a first region and a second region, the second region being located on a proximal side of the first region; a hub connected to a proximal portion of the tubular sheath; the first region including a first hydrophilic coating, and the second region including a groove portion extending from a distal side of the tubular sheath toward a proximal side of the tubular sheath, the groove portion including a second hydrophilic coating containing at least one of metal ions and a drug; a marker portion indicating a boundary portion between the first region and the second region on the tubular sheath; a strain relief portion covering a distal end of the hub; and wherein the groove portion includes a first groove portion located on the distal side relative to a distal end of the strain relief portion, the first groove portion provided with the second hydrophilic coating, and a second groove portion located on a proximal side of the distal end of the strain relief portion.

In accordance with another aspect, a method is disclosed of indwelling an introducer sheath into a body lumen, the method comprising: inserting the introducer sheath into the body lumen through a puncture site, the puncture site extending from a limb of a patient to a blood vessel wall, the introducer sheath including a tubular sheath, a hub connected to a proximal portion of the tubular sheath, and a strain relief portion, the tubular sheath including a first region and a second region, the second region being located on a proximal side of the first region, the first region having a first hydrophilic coating having lubricity, the second region including a groove portion extending from a distal side of the tubular sheath toward a proximal side of the tubular sheath, and the groove portion having a second hydrophilic coating containing at least one of metal ions and a drug; and positioning the second region over a length extending from the puncture site in the limb of the patient to the puncture site formed in the blood vessel wall to release the at least one of the metal ions and the drug retained by the second hydrophilic coating to the puncture site in the limb of patient and the blood vessel wall.

### BRIEF DESCRIPTION OF THE DRAWINGS

FIG. 1 is a view illustrating an introducer according to an embodiment.

FIG. 2 is a cross-sectional view of a medical elongated body according to the embodiment.

FIG. 3 is a cross-sectional view schematically illustrating a use example of the medical elongated body according to the embodiment.

FIG. 4 is a view illustrating a portion surrounded by a broken line portion 4A illustrated in FIG. 3 in an enlarged manner.

FIG. 5 is a cross-sectional view of a medical elongated body according to a modification.

### DETAILED DESCRIPTION

Set forth below with reference to the accompanying drawings is a detailed description of embodiments of a medical elongated body. Note that since embodiments described below are preferred specific examples of the present disclosure, although various technically preferable limitations are given, the scope of the present disclosure is not limited to the embodiments unless otherwise specified in the following descriptions.

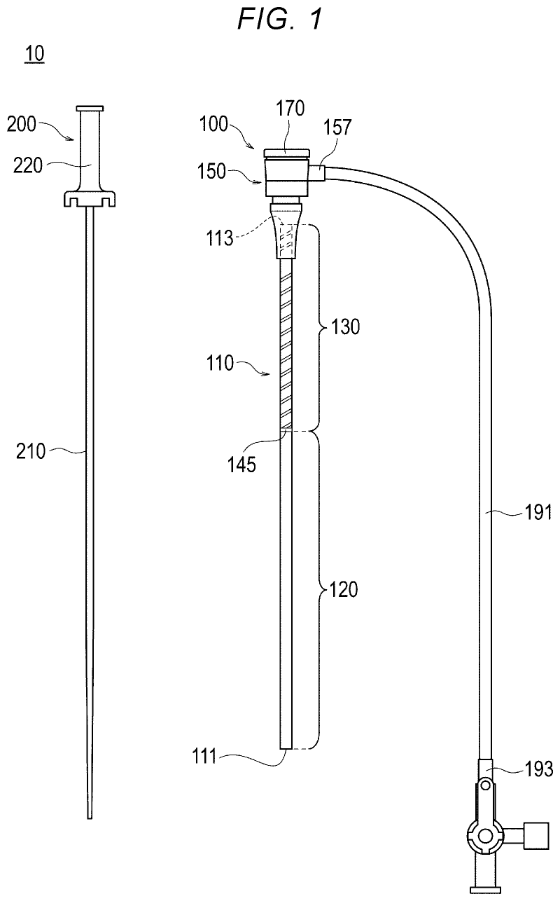

A medical elongated body according to the disclosure will be described with reference to FIGS. 1 to 4. In an embodiment, an example in which the medical elongated body according to the disclosure is applied to an introducer sheath 100 of an introducer 10 will be described.

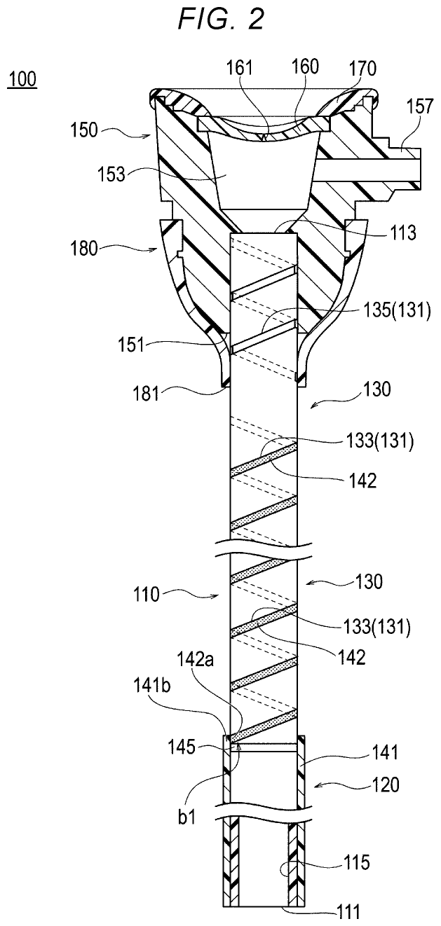

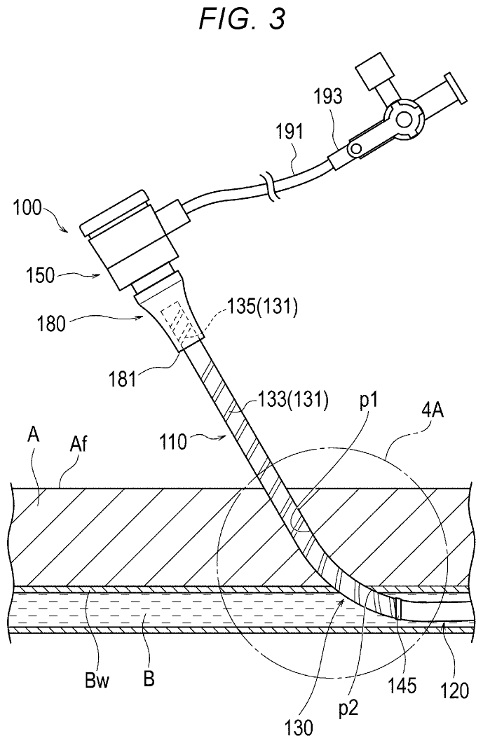

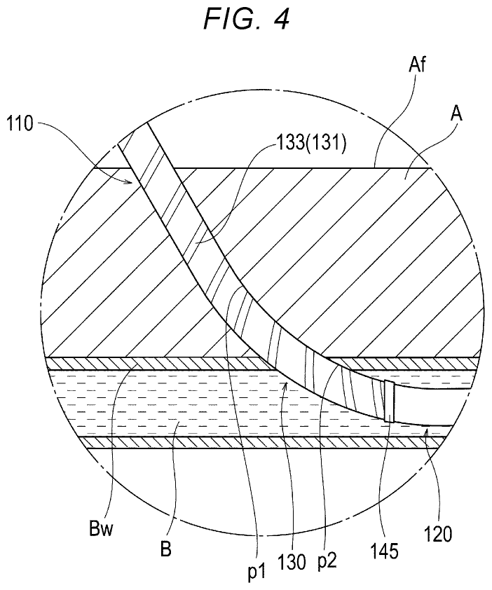

FIG. 1 is a view illustrating the introducer 10, FIG. 2 is a cross-sectional view (partial cross-sectional view along an axial direction) of the introducer sheath 100, and FIGS. 3 and 4 are cross-sectional views schematically illustrating a use example of the introducer sheath 100.

In the present disclosure, a side on which a hub 150 is disposed (an upper side in FIG. 1) in the introducer sheath 100 is referred to as a proximal side. In the introducer sheath 100, a side opposite to the proximal side and on which the introducer sheath 100 is introduced into a living body (a lower side in FIG. 1) is referred to as a distal side. A direction (a vertical direction in FIG. 1) in which a tubular sheath 110 (corresponding to a “catheter main body”) of the introducer sheath 100 extends is referred to as the axial direction. In the present disclosure, a distal portion means a certain range including a distal end (a most distal end) in the axial direction, and a proximal portion means a certain range including a proximal end (a most proximal end) in the axial direction.

### Introducer

As illustrated in FIG. 1, the introducer 10 includes the introducer sheath 100 and a dilator 200.

The introducer sheath 100 can be used to introduce, through a lumen 115 of the tubular sheath 110 (see FIG. 2), various medical instruments such as a dilator main body 210 of the dilator 200, a catheter device, and a guide wire into a blood vessel B (corresponding to a “living body lumen”).

### Introducer Sheath

As illustrated in FIGS. 1, 2, and 3, the introducer sheath 100 includes the tubular sheath 110 percutaneously inserted into the blood vessel B, the hub 150 connected to the proximal portion of the tubular sheath 110, and a strain relief portion 180 covering a distal end 151 of the hub 150.

As illustrated in FIG. 2, the tubular sheath 110 is implemented by a hollow member having the lumen 115 extending in the axial direction. A distal end 111 and a proximal end 113 of the tubular sheath 110 are each formed with an opening portion in communication with the lumen 115 and an outside of the lumen 115.

Examples of a constituent material for the tubular sheath 110 can include a polymer material such as polyolefin (for example, polyethylene, polypropylene, polybutene, an ethylene-propylene copolymer, an ethylene-vinyl acetate copolymer, an ionomer, or a mixture of at least two of the six), polyolefin elastomer, cross-linked polyolefin, polyvinyl chloride, polyamide, polyamide elastomer, polyester, polyester elastomer, polyurethane, polyurethane elastomer, fluororesin (for example, polytetrafluoroethylene, or a tetrafluoroethylene-ethylene copolymer), polycarbonate, polystyrene, polyacetal, polyimide, polyetherimide, or polyether ether ketone, or a mixture of the materials listed above.

As illustrated in FIGS. 1 and 2, the tubular sheath 110 includes a first region 120 and a second region 130 located on the proximal side in the axial direction relative to the first region 120. As illustrated in FIG. 1, a length of the second region 130 is shorter than a length of the first region 120. The second region 130 can be, for example, preferably 20 mm or more so as to be located over an entire length from a puncture site p1 formed in a limb A of a patient to a puncture site p2 formed in a blood vessel wall Bw. With this configuration, the tubular sheath 110 can release metal ions and a drug retained by a second hydrophilic coating of the second region 130 to a subcutaneous tissue in the vicinity of a puncture site.

The first region 120 has a first hydrophilic coating 141 having lubricity.

The second region 130 includes a groove portion 131 extending from the distal side of the tubular sheath 110 in the axial direction toward the proximal side.

The groove portion 131 can be implemented by a recess recessed from an outer surface of the tubular sheath 110 toward a central axis of the lumen 115. A depth of the groove portion 131 is not particularly limited as long as the depth is smaller than a thickness of a tube wall of the tubular sheath 110. A cross-sectional shape of the groove portion 131 may be, for example, a semicircular shape, a U-shape, and a rectangular shape, and is not limited to the semicircular shape, the U-shape, and the rectangular shape.

As illustrated in FIG. 2, the groove portion 131 includes a first groove portion 133 located on the distal side relative to a distal end 181 of the strain relief portion 180, and a second groove portion 135 located on the proximal side relative to the distal end 181 of the strain relief portion 180.

As illustrated in FIG. 2, the first groove portion 133 and the second groove portion 135 are not continuous in the axial direction of the tubular sheath 110. That is, in the tubular sheath 110, there is a region where no groove portion 131 is provided between the first groove portion 133 and the second groove portion 135.

At least a part of the groove portion 131 is provided with a second hydrophilic coating 142 containing at least one of the metal ions and the drug. The second hydrophilic coating 142 can be, for example, a hydrogel layer having the water swelling property, and the hydrogel layer immobilizes or retains the metal ions and the drug. In the present embodiment, the first groove portion 133 is provided with the second hydrophilic coating 142, and the second groove portion 135 is not provided with the second hydrophilic coating 142.

The first hydrophilic coating 141 may be implemented by a known material and a known method. As an example, the first hydrophilic coating 141 may be implemented by an acrylamide-based polymer material (for example, a block copolymer of polyacrylamide and polyglycidyl methacrylate-dimethylacrylamide (PGMA-DMAA)), polyvinylpyrrolidone, or hyaluronic acid.

The second hydrophilic coating 142 may be implemented by, for example, containing at least one of the metal ions and the drug in the material exemplified as the material for the first hydrophilic coating 141. Examples of the metal ions can include metal ions having an effect on early wound healing or hemostasis. Examples of the drug can include a hemostatic agent having an effect on hemostasis, such as ascorbic acid or sodium alginate. The second hydrophilic coating 142 can be immersed in, for example, a solution containing the metal ions and the hemostatic agent and then dried, whereby the metal ions and the hemostatic agent can be retained inside the second hydrophilic coating 142.

The second hydrophilic coating 142 can be preferably disposed, for example, in a state before the introducer sheath 100 is used, in a manner of not protruding from the outer surface of the tubular sheath 110. By disposing the second hydrophilic coating 142 in the first groove portion 133 in this manner, peeling of the second hydrophilic coating 142 can be suitably prevented when the outer surface of the tubular sheath 110 comes into contact with another member or the like and is scratched.

The groove portion 131 can extend spirally from the distal side toward the proximal side of the tubular sheath 110. In the present embodiment, the first groove portion 133 and the second groove portion 135 extend spirally. A spiral pitch, a spiral angle, a spiral winding direction, and the like of the groove portions 133 and 135 are not particularly limited. The groove portions 133 and 135 may be formed in shapes different from each other. For example, the first groove portion 133 may be formed in a spiral shape, and the second groove portion 135 may be formed in another shape.

As illustrated in FIGS. 1 and 2, the tubular sheath 110 includes a marker portion 145 indicating a distal end position of the second region 130. That is, the marker portion 145 indicates a boundary portion b1 between the first region 120 and the second region 130. Accordingly, a surgeon or the like can rather easily grasp a position of the second region 130 having the second hydrophilic coating 142.

In the present embodiment, as illustrated in FIG. 2, a proximal end 141b of the first hydrophilic coating 141 and a distal end 142a of the second hydrophilic coating 142 overlap each other. Therefore, the boundary portion b1 between the first region 120 and the second region 130 is a portion where the proximal end 141b of the first hydrophilic coating 141 and the distal end 142a of the second hydrophilic coating 142 overlap in the axial direction of the tubular sheath 110. In the present embodiment, the distal end 142a of the second hydrophilic coating 142 located at a distal end of the groove portion 131 is covered by the proximal end 141b of the first hydrophilic coating 141. Therefore, as illustrated in FIG. 2, the marker portion 145 is provided on the outer surface on the distal side relative to the distal end of the groove portion 131 of the tubular sheath 110 (the distal end 142a of the second hydrophilic coating 142) so as to indicate the distal end position of the second region 130. Accordingly, the marker portion 145 is covered with the first hydrophilic coating 141.

In the present embodiment, as illustrated in FIG. 2, the marker portion 145 can be formed by embedding a material having a predetermined color on the outer surface of the tubular sheath 110. The marker portion 145 may be formed on the tubular sheath 110 by printing a pattern having a predetermined color on the outer surface of the tubular sheath 110 or by disposing another member on the outer surface of the tubular sheath 110. A method for forming the marker portion 145 is not particularly limited.

The marker portion 145 may be implemented by, for example, a line-shaped marker having a predetermined color. A color, a shape, and the like of the marker portion 145 are not particularly limited. The marker portion 145 may be provided at a position on the outer surface that includes the boundary portion b1 of the tubular sheath 110 as long as the surgeon or the like can grasp the position of the second region 130. In this case, the marker portion 145 may be provided on a circumference where the groove portion of the tubular sheath 110 is provided.

As illustrated in FIG. 2, the hub 150 has an internal space 153 communicating with the lumen 115 of the tubular sheath 110.

A valve body 160 is disposed in the internal space 153 of the hub 150. The valve body 160 helps prevent blood that flowed into the lumen 115 of the tubular sheath 110 from leaking to the outside of the hub 150.

The valve body 160 can be implemented by an elastic member formed with a slit 161 through which the dilator main body 210 is insertable. The valve body 160 may have, for example, a substantially elliptical disc shape. The valve body 160 is fixed to the hub 150 by a predetermined cap 170. A specific shape, a material, a slit structure, and the like of the valve body 160 are not particularly limited.

The cap 170 is fitted to a proximal portion of the hub 150 in a manner of surrounding a part of an outer peripheral surface of the hub 150. The cap 170 may have, for example, a structure of being fixed to the hub 150 inside the hub 150. The invention is not limited to fitting, and the cap 170 may also be fixed to the hub 150 by, for example, screwing or adhering.

The hub 150 is provided with a side port 157 communicating with the internal space 153 of the hub 150. The side port 157 may be connected with one end portion of a tube 191 (see FIG. 1). The other end portion of the tube 191 may be connected with, for example, a three-way stopcock 193.

The strain relief portion 180 helps prevent kink, breakage, or the like in the tubular sheath 110. As illustrated in FIG. 2, the strain relief portion 180 can be externally fitted to the tubular sheath 110 and the hub 150. A distal portion of the strain relief portion 180 is disposed in a manner of surrounding a certain range of the proximal portion of the tubular sheath 110. A proximal portion of the strain relief portion 180 is disposed in a manner of surrounding a certain range on a distal side of the hub 150.

### Dilator

As illustrated in FIG. 1, the dilator 200 includes the dilator main body 210 insertable into the lumen 115 of the tubular sheath 110 of the introducer sheath 100, and a dilator hub 220 that is fixed to a proximal side of the dilator main body 210.

The dilator 200 can be used to help prevent the tubular sheath 110 from breakage and to widen the puncture sites p1 and p2 (see FIG. 3) when the tubular sheath 110 of the introducer sheath 100 is inserted into the blood vessel B.

Next, a use example of the introducer sheath 100 will be described with reference to FIGS. 3 and 4.

FIGS. 3 and 4 illustrate a state in which the tubular sheath 110 of the introducer sheath 100 is inserted into the blood vessel B through the puncture site p1 formed in the limb A (for example, a forearm) of the patient and the puncture site p2 formed in the blood vessel wall Bw.

In treatment using the introducer 10, the surgeon or the like inserts the first region 120 and the second region 130 of the tubular sheath 110 into the blood vessel B as illustrated in FIG. 4. When the surgeon or the like inserts the second region 130 into the blood vessel B, the blood enters the first groove portion 133 formed in the second region 130. The blood that entered the first groove portion 133 moves toward the proximal end of the tubular sheath 110 along the first groove portion 133. When the blood comes into contact with the second hydrophilic coating 142 disposed in the first groove portion 133, the metal ions and the drug contained in the second hydrophilic coating 142 is released. The first groove portion 133 extends spirally in the axial direction of the tubular sheath 110. Therefore, the metal ions and the drug is released from each part of the first groove portion 133 toward the periphery of the tubular sheath 110. Therefore, the surgeon or the like can give a drug effect of the metal ions and the drug to the vicinity of a skin surface Af in the vicinity of the puncture site p1 formed in the limb A, the subcutaneous tissue surrounding the puncture site p1, and the vicinity of the puncture site p2 of the blood vessel wall Bw.

When the second hydrophilic coating 142 contains the metal ions, the metal ions released from the first groove portion 133 act on the puncture sites p1 and p2, and contribute to angiogenesis, collagen production, activity of blood coagulation factors, and the like, thereby contributing to early wound healing and promotion of a hemostatic effect at the puncture sites p1 and p2. In addition, the metal ions also contribute to, by an antibacterial action of the metal ions, prevention of infection diseases through the puncture sites p1 and p2. When the second hydrophilic coating 142 contains the hemostatic agent as the drug, the hemostatic agent released from the first groove portion 133 acts on the puncture sites p1 and p2 and contributes to promotion of blood coagulation or the like, thereby contributing to promotion of the hemostatic effect at the puncture sites p1 and p2.

In the tubular sheath 110, the first groove portion 133 and the second groove portion 135 are not continuous in the axial direction of the tubular sheath 110 (see FIG. 2). Therefore, the blood moving along the first groove portion 133 can be prevented from reaching the second groove portion 135. Accordingly, the blood can be prevented from flowing into the inside of the strain relief portion 180 or the internal space 153 of the hub 150.

As described above, the introducer sheath 100 according to the present embodiment includes the tubular sheath 110 configured to be percutaneously inserted into the blood vessel B, and the hub 150 connected to the proximal portion of the tubular sheath 110. The tubular sheath 110 includes the first region 120 and the second region 130 located on the proximal side relative to the first region 120. The first region 120 has the first hydrophilic coating 141 having lubricity. The second region 130 includes the groove portion 131 extending from the distal side of the tubular sheath 110 toward the proximal side. The groove portion 131 has the second hydrophilic coating 142 containing at least one of the metal ions and the drug.

In the introducer sheath 100 configured as described above, the second hydrophilic coating 142 containing at least one of the metal ions and the drug is located in the groove portion 131 formed in the second region 130 of the tubular sheath 110. When the tubular sheath 110 is inserted into the blood vessel B, the blood enters the groove portion 131 of the second region 130, the second hydrophilic coating 142 swells, and thus the introducer sheath 100 can release the metal ions and the drug retained by the second hydrophilic coating 142 to a living body tissue (for example, the subcutaneous tissue) and the blood vessel wall Bw surrounding the tubular sheath 110. The second hydrophilic coating 142 is located in the groove portion 131 of the second region 130. Therefore, when the surgeon or the like operates the introducer sheath 100, since fingers, surrounding articles, and the like do not come into contact with the second hydrophilic coating 142, the introducer sheath 100 can help prevent the peeling, from the tubular sheath 110, of the second hydrophilic coating 142 that retains the metal ions and the drug. Accordingly, the introducer sheath 100 can simplify an operation of disposing the second hydrophilic coating 142 on the puncture sites p1 and p2 by the surgeon or the like, and thus the metal ions and the drug retained by the second hydrophilic coating 142 can be rather easily provided on the puncture sites p1 and p2 of a patient. Since the second hydrophilic coating 142 is located in the groove portion 131, the introducer sheath 100 can help prevent the tubular sheath 110 from slipping when the surgeon or the like grips the proximal side of the tubular sheath 110 with fingers or the like. In addition, the introducer sheath 100 can also help prevent, when the surgeon or the like indwells the second region 130 having the second hydrophilic coating 142 at the puncture sites p1 and p2, a movement of the tubular sheath 110 (sliding of the tubular sheath 110 due to the second hydrophilic coating 142) at an indwelling site. In the introducer sheath 100, flexibility of the tubular sheath 110 can be improved by the groove portion 131 provided in the second region 130 of the tubular sheath 110. Therefore, the introducer sheath 100 can help prevent the kink in the tubular sheath 110 while the tubular sheath 110 is indwelled in the blood vessel B. In the introducer sheath 100, since the first region 120 located on the distal side of the tubular sheath 110 relative to the second region 130 has the first hydrophilic coating 141, the tubular sheath 110 can be rather smoothly inserted into the blood vessel B, and the second hydrophilic coating 142 located in the second region 130 can be rather smoothly disposed in the puncture sites p1 and p2.

The tubular sheath 110 includes the marker portion 145 indicating the boundary portion b1 between the first region 120 and the second region 130. Therefore, the surgeon or the like can rather easily visually grasp the distal end position of the second region 130. Accordingly, when the tubular sheath 110 is inserted into the blood vessel B, the surgeon or the like can rather easily grasp the distal end of the groove portion 131. Therefore, the surgeon or the like can reliably dispose the groove portion 131 in the blood vessel B, and can release the metal ions and the drug retained by the second hydrophilic coating to the puncture sites p1 and p2 of the patient.

The groove portion 131 extends spirally from the distal side of the tubular sheath 110 toward the proximal side. In the introducer sheath 100, the flexibility of the second region 130 located on the proximal side of the tubular sheath 110 in a circumferential direction can be improved by the groove portion 131 extending spirally. Therefore, the tubular sheath 110 can help prevent the kink in the tubular sheath 110 while the tubular sheath 110 is indwelled in the blood vessel B. The introducer sheath 100 can efficiently release the metal ions and the drug retained by the second hydrophilic coating 142 over entire circumferences of the puncture sites p1 and p2 by the groove portion 131 extending spirally. The introducer sheath 100 facilitates, by the groove portion 131 extending spirally, the movement of the blood from the distal side to the proximal side along the groove portion 131.

The introducer sheath 100 includes the strain relief portion 180 that covers the distal end 151 of the hub 150. The groove portion 131 includes the first groove portion 133 located on the distal side relative to the distal end 181 of the strain relief portion 180 and provided with the second hydrophilic coating 142, and the second groove portion 135 located on the proximal side relative to the distal end 181 of the strain relief portion 180 and not provided with the second hydrophilic coating 142. Therefore, the introducer sheath 100 can help prevent the blood moving along the first groove portion 133 from reaching the second groove portion 135 while improving the flexibility of the second region 130 located on the proximal side of the tubular sheath 110 in the circumferential direction. Accordingly, the blood can be prevented from flowing into the inside of the strain relief portion 180 or the internal space 153 of the hub 150. Since the introducer sheath 100 is provided with the second groove portion 135, differences in physical properties at a connecting portion between the tubular sheath 110 and the hub 150 can be alleviated, and the flexibility at the proximal portion of the introducer sheath 100 can be improved.

The proximal end 141b of the first hydrophilic coating 141 and the distal end 142a of the second hydrophilic coating 142 overlap each other. Therefore, in the introducer sheath 100, a region where no hydrophilic coating is provided is not formed between the first hydrophilic coating 141 and the second hydrophilic coating 142. Accordingly, the introducer sheath 100 can further improve insertability of the tubular sheath 110 into the blood vessel B, and the second hydrophilic coating 142 located in the second region 130 can be smoothly disposed at the puncture sites p1 and p2. In the introducer sheath 100, since the first hydrophilic coating 141 and the second hydrophilic coating 142 overlap each other, when the surgeon or the like inserts the first hydrophilic coating 141 of the tubular sheath 110 into the blood vessel B, the first hydrophilic coating 141 absorbs body fluid such as the blood, and the second hydrophilic coating 142 can be swollen by a capillary phenomenon or the like. Therefore, the introducer sheath 100 can efficiently release, through the second hydrophilic coating 142, the metal ions and the drug retained by the second hydrophilic coating 142 to the puncture sites p1 and p2.

Next, a modification will be described. In the modification, duplicated descriptions about contents already described will be omitted. Contents not particularly described in the modification may be the same as those in the embodiment described above.

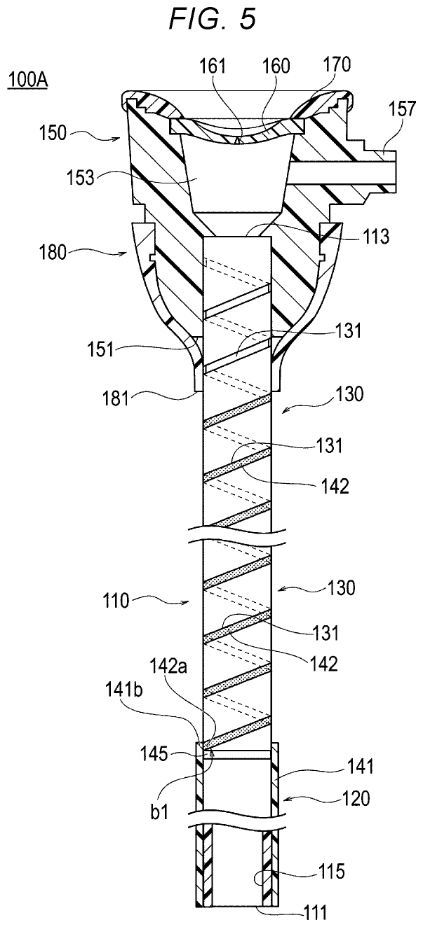

FIG. 5 illustrates an introducer sheath 100A according to the modification.

In the introducer sheath 100A according to the modification, the groove portion 131 is formed from the distal side relative to the distal end 181 of the strain relief portion 180 toward the proximal side relative to the distal end 181 of the strain relief portion 180. That is, in the introducer sheath 100A according to the modification, in the groove portion 131, a portion formed on the distal side relative to the distal end 181 of the strain relief portion 180 and a portion formed on the proximal side relative to the distal end 181 of the strain relief portion 180 are formed continuously. According to the introducer sheath 100A configured in this way, since the groove portion 131 continuously extends along the axial direction of the second region 130 of the tubular sheath 110, the flexibility is improved over an entire range of the second region 130 in the axial direction.

Although the medical elongated body according to the disclosure has been described above through the embodiment and the modification, the disclosure is not limited only to the configurations described above, and can be appropriately changed based on the description of the claims

An application target of the medical elongated body is not limited only to an introducer sheath, and may be applied to various catheters including a catheter main body in which the first region and the second region are provided, an outer tube, or the like. Details of a specific procedure using the medical elongated body, treatment procedures, and the like are not particularly limited.

The structure of each part, the arrangement of members, and the like described in the disclosure can be appropriately changed, and it is possible to omit the use of additional members described with reference to the drawings, and to use other additional members and the like appropriately.

The detailed description above describes embodiments of a medical elongated body. These disclosed embodiments represent examples of the medical elongated body disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A medical elongated body comprising:
a catheter main body configured to be percutaneously inserted into a living body lumen;
a hub connected to a proximal portion of the catheter main body;
the catheter main body including a first region and a second region, the second region being located on a proximal side of the first region;
the first region having a first hydrophilic coating having lubricity;
the second region including a groove portion extending from a distal side of the catheter main body toward a proximal side of catheter main body; and
the groove portion having a second hydrophilic coating containing at least one of metal ions and a drug.

2. The medical elongated body according to claim 1, wherein the catheter main body includes a marker portion indicating a boundary portion between the first region and the second region.

3. The medical elongated body according to claim 1, wherein the groove portion extends spirally from the distal side of the catheter main body toward the proximal side of the catheter main body.

4. The medical elongated body according to claim 1, further comprising:
a strain relief portion covering a distal end of the hub; and
wherein the groove portion includes a first groove portion located on a distal side relative to a distal end of the strain relief portion, the first groove portion provided with the second hydrophilic coating, and a second groove portion located on a proximal side relative to the distal end of the strain relief portion, and wherein the second groove portion is not provided with the second hydrophilic coating.

5. The medical elongated body according to claim 1, further comprising:
a strain relief portion covering a distal end of the hub; and
wherein the groove portion is formed from a distal side relative to a distal end of the strain relief portion toward a proximal side relative to the distal end of the strain relief portion.

6. The medical elongated body according to claim 1, wherein a proximal end of the first hydrophilic coating and a distal end of the second hydrophilic coating overlap each other.

7. The medical elongated body according to claim 1, wherein the drug is a hemostatic agent.

8. The medical elongated body according to claim 1, wherein a length of the second region in an axial direction is shorter than a length of the first region in the axial direction.

9. The medical elongated body according to claim 8, wherein the length of the second region is at least 20 mm.

10. The medical elongated body according to claim 1, wherein the groove portion is a recess recessed from an outer surface of the catheter main body toward a central axis of a lumen of the catheter main body.

11. The medical elongated body according to claim 1, wherein the groove portion has a cross-sectional shape selected from a semicircular shape, a U-shape, and a rectangular shape.

12. The medical elongated body according to claim 4, wherein the first groove portion and the second groove portion are not continuous in an axial direction of the catheter main body.

13. The medical elongated body according to claim 1, wherein the second hydrophilic coating is a hydrogel layer having a water swelling property, and wherein the hydrogel layer is configured to immobilize or retain the at least one of metal ions and the drug.

14. The medical elongated body according to claim 1, wherein a proximal end of the first hydrophilic coating and a distal end of the second hydrophilic coating overlap each other in an axial direction of the catheter main body.

15. An introducer sheath comprising:
a tubular sheath, the tubular sheath including a first region and a second region, the second region being located on a proximal side of the first region;
a hub connected to a proximal portion of the tubular sheath;
the first region including a first hydrophilic coating, and the second region including a groove portion extending from a distal side of the tubular sheath toward a proximal side of the tubular sheath, the groove portion including a second hydrophilic coating containing at least one of metal ions and a drug;
a marker portion indicating a boundary portion between the first region and the second region on the tubular sheath;
a strain relief portion covering a distal end of the hub; and
wherein the groove portion includes a first groove portion located on a distal side relative to a distal end of the strain relief portion, the first groove portion provided with the second hydrophilic coating, and a second groove portion located on a proximal side of the distal end of the strain relief portion.

16. The introducer sheath according to claim 15, wherein a proximal end of the first hydrophilic coating and a distal end of the second hydrophilic coating overlap each other.

17. A method of indwelling an introducer sheath into a body lumen, the method comprising:
inserting the introducer sheath into the body lumen through a puncture site, the puncture site extending from a limb of a patient to a blood vessel wall, the introducer sheath including a tubular sheath, a hub connected to a proximal portion of the tubular sheath, and a strain relief portion, the tubular sheath including a first region and a second region, the second region being located on a proximal side of the first region, the first region having a first hydrophilic coating having lubricity, the second region including a groove portion extending from a distal side of the tubular sheath toward the proximal side of the tubular sheath, and the groove portion having a second hydrophilic coating containing at least one of metal ions and a drug; and
positioning the second region over a length extending from the puncture site in the limb of the patient to the puncture site formed in the blood vessel wall to release the at least one of the metal ions and the drug retained by the second hydrophilic coating to the puncture site in the limb of patient and the blood vessel wall.

18. The method according to claim 17, wherein the groove portion includes a first groove portion located on a distal side relative to a distal end of the strain relief portion, the first groove portion provided with the second hydrophilic coating, and a second groove portion located proximally of the distal end of the strain relief portion, and wherein the at least one of the metal ions and the drug is released from the first groove portion toward a periphery of the tubular sheath.

19. The method according to claim 18, wherein the first groove portion and the second groove portion are not continuous in the axial direction of the tubular sheath such that blood moving along the first groove portion does not reach the second groove portion.

20. The method according to claim 18, wherein the metal ions released from the first groove portion act on the puncture site in the limb of patient and the blood vessel wall to contribute to one or more of angiogenesis, collagen production, activation of blood coagulation factors, an antibacterial action, and prevention of infection diseases.
